(19) United States
(12) Patent Application Publication
(10) Pub. No.: US 2006/0259127 A1
(43) Pub. Date: Nov. 16, 2006

(54) Apparatus and method for anchoring endoluminal prostheses in tortuous geometries

(76) Inventors: Deborah Tolomeo, San Jose, CA (US); Jason Tolomeo, San Jose, CA (US)

Correspondence Address: JASON TOLOMEO, 621 PARMA WAY, LOS ALTOS, CA, 94024, US

(21) Appl. No.: 11/125,641

(22) Filed: May 10, 2005

(51) Int. Cl. A61F 2/06

(52) U.S. Cl. 623/1.16

(57) ABSTRACT

The present invention is an endoluminal prothesis consisting of one or more anchor bodies and a main body for the treatment of vascular disease, especially when such disease requires the ability to anchor in a region remote from the diseased tissue. Anchor bodies and main bodies attach together in a manner to allow significant relative rotation between bodies and to accommodate changes in lumen tortousity with time. The device has numerous advantages in the treatment of damaged vasculature. Methods of deploying a stent, stent-graft, or filtering system constructed in accordance with the present invention are also provided.

### CROSS REFERENCE TO RELATED APPLICATION

Not applicable.

### STATEMENT REGARDING FEDERALLY SPONSORED RESEARCH OR DEVELOPMENT.

Not Applicable

### BACKGROUND OF THE INVENTION

1. Field of the Invention

The present invention relates generally to the field of minimally invasive techniques using endoluminal prostheses, such as stents, grafts, stent-grafts, filters, and the like for treatment of arterial or venous disease, including the repair of aneurysms and prevention of embolic events. More particularly, the present invention provides methods, systems, and devices for anchoring an endoluminal prosthesis, particularly where the lumen to be treated does not have sufficient landing area for anchoring the endoluminal prosthesis at the proximal or distal portion. The present invention also provides methods, systems and devices for anchoring an endoluminal prosthesis where the vasculature is tortuous near the diseased region, or the vasculature changes tortuosity with time.

2. Background of the Invention

A number of medical procedures involve or can be supplemented with the placement of an endoluminal prosthesis, such as a stent, graft, stent-graft, filter, or combination thereof. These endoluminal prostheses, commonly referred to as devices, can be implanted in a lumen, such as a blood vessel or other natural pathway of a patient's body. In order to provide delivery to the treatment location, such devices are typically placed in a small diameter delivery system and then advanced through the body lumen to the site where the device is to be placed. Upon delivery to the treatment location, known devices employ techniques such as plastic deformation, elastic recovery, superelastic recovery, thermal recovery, or balloon expansion to expand the device to a larger diameter. This expansion allows the device to engage the inner wall of the body lumen and anchor the device. In addition, the device may engage the inner wall of the body lumen with hooks or barbs.

A disadvantageous characteristic that many prostheses have in common is a requirement of a region of healthy tissue in which to place the device so as to provide appropriate anchoring forces. Of particular relevance for the present invention, is treatment of vascular aneurysms and treatment of occlusive disease.

Vascular aneurysms are an abnormal dilation of a blood vessel, resulting from a weakening of the arterial wall due to disease or genetic predisposition. Vascular aneurysms can occur in any blood vessel, although most occur in the aorta and peripheral arteries. The majority of aortic aneurysms occur in the abdominal aorta (commonly referred to as “abdominal aortic aneurysms”), usually beginning below the renal arteries and often extending into one or both of the iliac arteries. Aneurysms also occur in the thoracic aorta, usually beginning near the aortic arch, and extending peripherally to the renal arteries. All aneurysms, in both the aortic and peripheral arteries exhibit the possibility of rupture when the vessel wall becomes too thin to tolerate vascular loading. In the case of thoracic and abdominal aortic aneurysms, rupture is often fatal.

Treatment of an aneurysm occurring near a branched region presents numerous difficulties in device design. This is because the zone of healthy tissue between the branching arteries and the aneurysm is reduced by the progression of the disease. In advanced cases the aneurysm may be so large as to have almost reached the branched arteries, thereby providing an anchoring region that is too small to sufficiently anchor the device. Therefore, it would be desirable to have a device that could anchor in a region of healthy tissue remote from the diseased region.

In addition, progression of disease may cause increased tortuosity or angulations of the aneurysm relative to the anchoring region. This change in vascular orientation makes anchoring the device difficult, because the increased tortuosity or angulation tends to pull and twist the device. In some cases this can result in dislodgement of the device from the anchoring region. Therefore, it would be desirable to have a device, which could accommodate changes in vessel orientation while maintaining adequate anchoring forces.

Similar phenomena occur in occlusive disease. In occlusive disease, the vessel becomes narrow as a result of the build-up of plaque and other occlusive materials. For example, in occlusive disease occurring in the iliac artery, a stenosis, or narrowing of the artery may occur near the bifurcation of the common iliac. Placement of a device in this region can be difficult because the disease state is such that placement of a stent or stent-graft in the common iliac or external iliac may prevent blood flow to the internal iliac. Therefore, it would be desirable to have a device that could anchor in a region of healthy tissue remote from the diseased region.

Arterial or venous disease can also occur in regions of high curvature, where placement and anchoring of a device can be particularly difficult. For example, in the thoracic arch, it can be very difficult to place a device with sufficient anchoring force. Similarly, placement of arterial or venous filters can also occur in regions of high curvature.

In view of the foregoing, it would be desirable to provide a prosthetic device and method of deploying and anchoring a device that overcomes drawbacks of previously known stent, stent-graft, and filtering systems. It is an object of the present invention to provide a device and method that can anchor in a region of healthy tissue that is remote from the disease location. It is a further object of the present invention to provide a device and method that is readily deployable in tortuous vessels where the region to be treated is in a region of high curvature. Moreover, it is an object of the present invention to provide a device and method that accommodates changes in the tortuosity of the vessel over time. In addition, it is an object of the present invention to provide a device and method that can be deployed in sequence, whereby the anchoring body is deployed prior to positioning of the main body, which may comprise a stent, stent-graft, filter, or other device. Moreover, it is an object of the present invention to provide a device and method with a first body that may be deployed in a vessel having a large diameter, and a second body that may be deployed in a branch vessel having a smaller diameter or vice versa.

It is a further object of the present invention to provide a device capable of being deployed in a bifurcated or branched vessel that enables a first body of the anchoring system to be deployed in a trunk vessel having a first longitudinal axis, and a second body of the anchoring system to be deployed in one or more branch vessels having longitudinal axes forming an angle with the first longitudinal axis.

It is a further object of the present invention to provide endoluminal prostheses which accept variations in geometry along body lumens without compromising their therapeutic effectiveness. It is a further object of the present invention to provide a device and methods for its placement which would allow flow to side branches and bifurcated regions while providing sufficient anchoring force to support the device. It is a further object to provide adaptable prostheses and methods for their placement which would facilitate effective treatment of widely varying luminal system geometries without requiring an excessive inventory of prostheses to chose from.

### SUMMARY OF THE INVENTION

The present invention is an endoluminal prothesis consisting of an anchor body and a main body for the treatment of vascular disease, especially when such disease requires the ability to anchor in a region remote from the diseased tissue. Methods of deploying a stent, stent-graft, or filtering system constructed in accordance with the present invention are also provided.

### BRIEF DESCRIPTION OF THE DRAWINGS

The foregoing and other objects and advantages of the invention will be appreciated more fully from the following description thereof, with reference to the accompanying drawings wherein:

FIG. 1 Illustrates an embodiment of the anchor body of the present invention.

FIG. 2 Illustrates an embodiment of the main body.

FIG. 3 Illustrates a combination of the anchor body and the main body where the connection between the two bodies effectively forms a ball joint.

FIG. 4. Illustrates an embodiment of the present invention deployed within a bifurcated vessel.

FIG. 5. Illustrates an embodiment of the invention deployed within a vessel where the anchor body is located at a region of relatively healthy tissue remote to the diseased area to be treated by the main body section in a region of highly tortuous geometry that may change over time.

FIG. 6. Vessel with anchor body and main body that allows flow to side branch vessel.

FIG. 7. Illustrates an anchor body which can accommodate multiple main bodies for use as a multiple branch stent-graft.

FIG. 8 Illustrates an intermediate anchor body capable of forming ball joint like links at either end to be used for multiple anchor bodies or main bodies in series.

FIG. 9. Illustrates multiple anchor bodies attached in a serial fashion to provide additional anchoring.

### DETAILED DESCRIPTION OF THE INVENTION

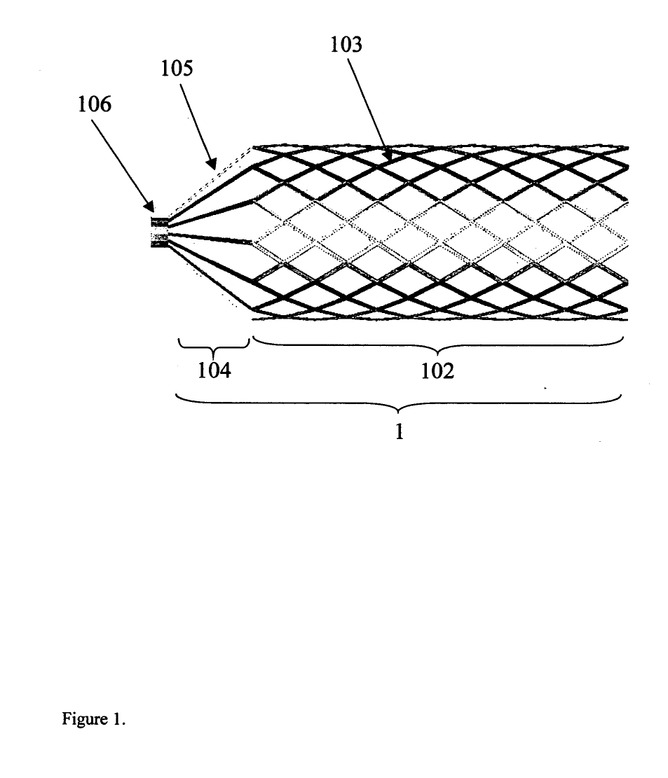

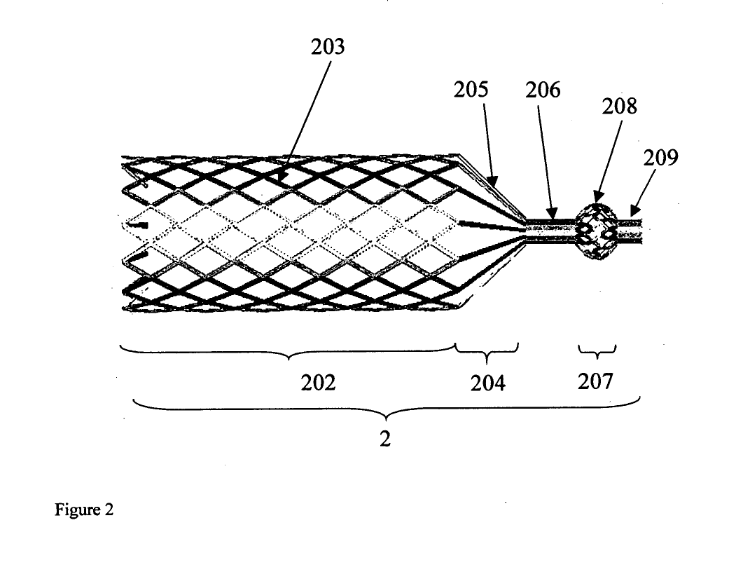

In the preferred embodiment the device consists of two separate bodies, an anchor body (1) as shown in FIG. 1 and a main body (2) as shown in FIG. 2. Said anchor body (1) and said main body (2) may be deployed into the lumen in sequence or may be deployed simultaneously.

An anchor body (1) is shown in FIG. 1. It consists of a long cylindrical section (102) formed of a lattice of thin diagonal members (103) that can expand in the radial direction and transitions at one end to a short tapered section (104). The design and pattern of the lattice of diagonal members (103) depends on the application, material, and stiffness desired. One common device geometry comprises a lattice of diamond-shaped elements which are joined in a ring. These diamond-shaped elements are circumferentially expandable as the prosthesis is deployed from the small profile configuration to the large profile configuration. Other common geometries include helically wound wires and filaments, zig-zag rings, braided filaments, woven helical filaments and the like. These geometries may be connected together by connectors or bridges of a straight or sinuous form. This lattice of structural elements is designed to accommodate radial expansion from a small profile configuration to a large profile configuration. In the case of a tapered, branched, or bifurcated lumen, the device may be designed to expand to different diameters at different points along the device length. And, in the case of longer device lengths, the network or lattice of structural elements may be connected together by connectors residing along the outer structural frame. Depending upon the bridge design, the bridge may allow the prosthesis to conform to a more tortuous anatomy during delivery, or implantation, or both. In the preferred embodiment, they are simply shown as a diamond pattern. Utilization of alternate patterns such as “U,” “V”, “W”, or “S” shapes that provide the ability for radial expansion will be apparent to one skilled in the art.

The tapered section (104) is made of thin longitudinal members (105) and terminates in a straight tube section (106). In the preferred embodiment, the straight tube section (106) is a section of the original tube stock that the entire anchor body (1) is made from. In an alternate embodiment, longitudinal members (105) can be similar in form to diagonal members (103) so long as they taper to straight tube section (106). In a further alternate embodiment, straight tube section (106) could have the same cut pattern as cylindrical section (102) but has not been fully expanded so that in the deployed configuration it remains at a reduced radius from cylindrical section (102).

The main body (2) is shown in FIG. 2. It consists of a long cylindrical section (202) formed of a lattice of thin diagonal members (203) that can expand in the radial direction and transitions at one end to a short tapered section (204). The design and pattern of the lattice of diagonal members (203) depends on the application, material, and stiffness desired. In the preferred embodiment, they are simply shown as a diamond pattern, but utilization of alternate patterns to provide the ability for radial expansion will be apparent to one skilled in the art.

Tapered section (204) is made of thin longitudinal members (205) and terminates in an intermediate straight tube section (206) that is a section of the original tube stock that the entire anchor body (1) is made from. In an alternate embodiment, longitudinal members (205) can be similar in construction to diagonal members (203) so long as they taper to straight tube section (206). In a further alternate embodiment, tube section (206) could have the same diagonal cut pattern as cylindrical section (202) but has not been treated to expand so that in the deployed configuration it remains at a reduced radius from cylindrical section (202). The intermediate tube section (206) transitions to a partially expanded spherical section (207) made of a lattice of slender diagonal members (208) that can be expanded in the radial direction. In an alternate embodiment the partially expanded section (207) could be oval in shape and mate with an oval shaped tube section (106). In this alternate embodiment, this would reduce the rotational freedom of the interface about the long axis of the device and therefore prescribe the angular orientation between the two. Radiopaque markers could then be used to verify an certain angular orientation at deployment. Again, the pattern of the diagonal members (208) can be varied and depends on the application, material, and desired stiffness. In the preferred embodiment they are simply shown as a diamond pattern, but utilization of alternate patterns such as “U,” “V”, “W”, or “S” shapes to provide the ability for radial expansion will be apparent to one skilled in the art.

Spherical section (207) transitions into an end straight tube section (209). End straight tube section (209) may have cut slots in it giving a diagonal brace pattern, but it is substantially unexpanded and is most conveniently the radius of the original tube stock from which the device is manufactured.

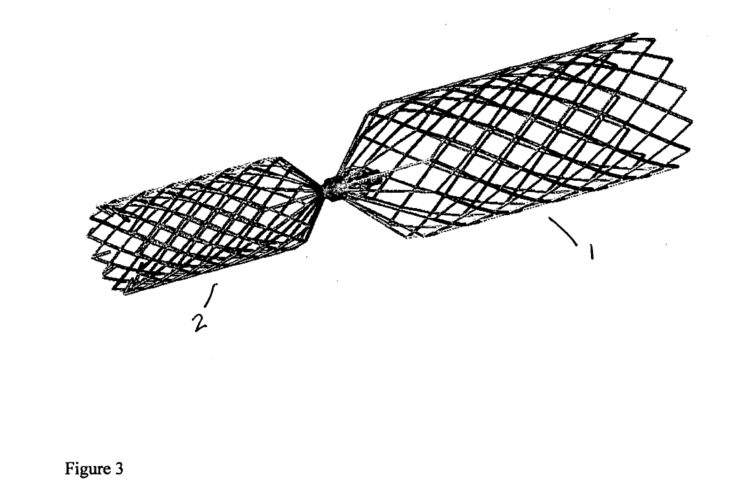

In the preferred embodiment, anchor body (1) is installed through a catheter and deployed into the vessel at a location of relatively healthy tissue that is proximal to the damaged tissue. Once the Anchor body is satisfactorily located and deployed for example via a catheter delivery system, Main body (2) is inserted into Anchor body (1) and then the spherical section (207) is allowed to expand within Anchor body (1) as shown in FIG. 3. The delivery system is removed and the Main body (2) is held in place in part by contact with the vessel wall but more substantially by the reinforcing action of spherical section (207) trapped within or beyond tube section (106). When the spherical section (207) is contained within or beyond the tube section (106) it acts substantially as a joint which prevents relative lateral translation of the main body (2) and the anchor body (1) but allows relative rotation between the main body (2) and the anchor body (1). Anchor body (1) thereby holds Main body (2) in place through the contact at trapped spherical section (207), even in the absence of direct adequate friction between Vessel Wall and Main body (2). In an alternate embodiment, Main body (2) is inserted into Anchor body (1) prior to deployment while still in the delivery system. The two bodies are then delivered and located into the vessel at the appropriate locations simultaneously and then allowed to expand, trapping spherical section (207) within tube section (106).

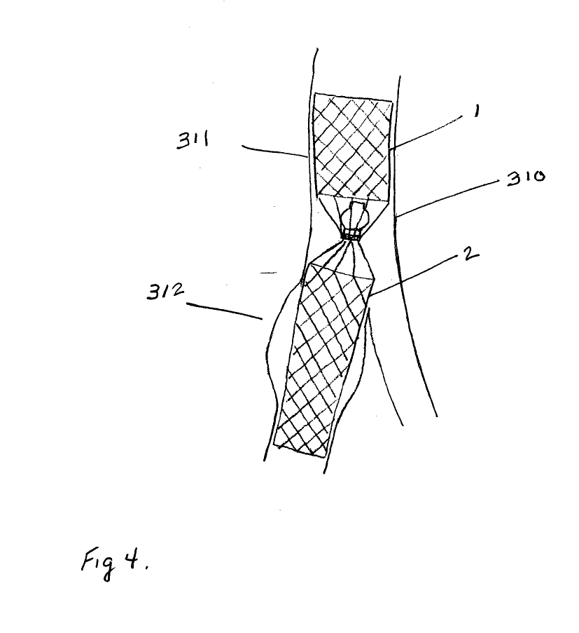

In use with a bifurcated vessel (310), as shown in FIG. 4 anchor body (1) is deployed above the bifurcation in a region of healthy tissue (311) that can support the necessary anchor load. Main body (2) is deployed to cover the region of damaged tissue (312).

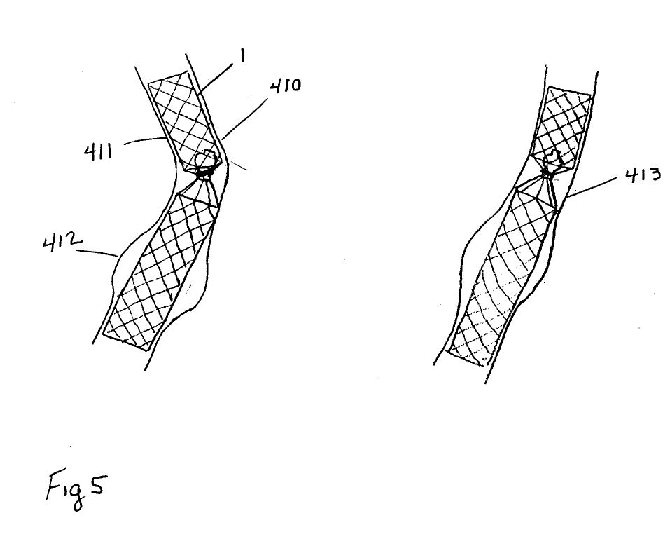

In use with a tortuous geometry (410) as shown in FIG. 5, anchor body (1) is deployed above the damaged tissue in a region of relatively healthy tissue (411) that can support the necessary anchor load. Main body (2) is deployed to cover the area of damaged tissue (312). Changes in angulation and tortuosity (413) are accommodated by the ball-joint like connection between anchor body (1) and main body (2). Changes in tortuosity may be rapidly or slowly varying with time.

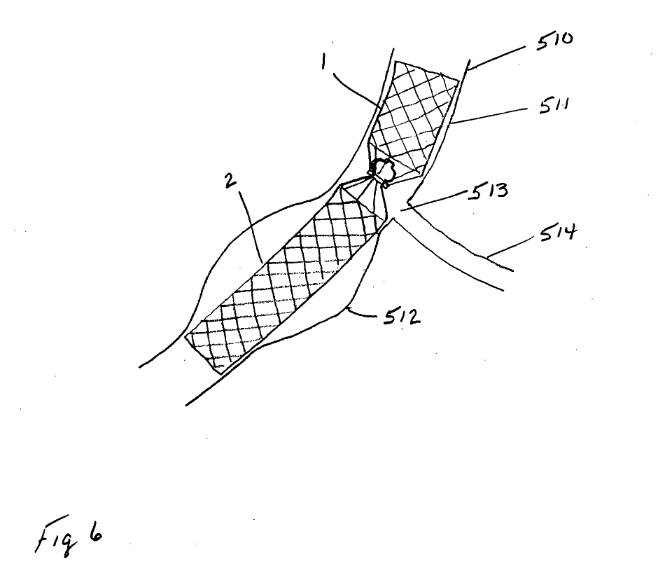

In the application to vessels with side branches (510) as shown in FIG. 6, anchor body (1) is deployed above the side branch (514) in a region of relatively healthy tissue (511) that can support the necessary anchor loads. Main body (2) is deployed to cover the region of damaged tissue (512). The connection between anchor body (1) and main body (2) occurs at a region of reduced diameter. This reduced diameter region of the device is located adjacent to the entrance to the branch vessel (513) so that flow to/from the branch vessel is not obstructed.

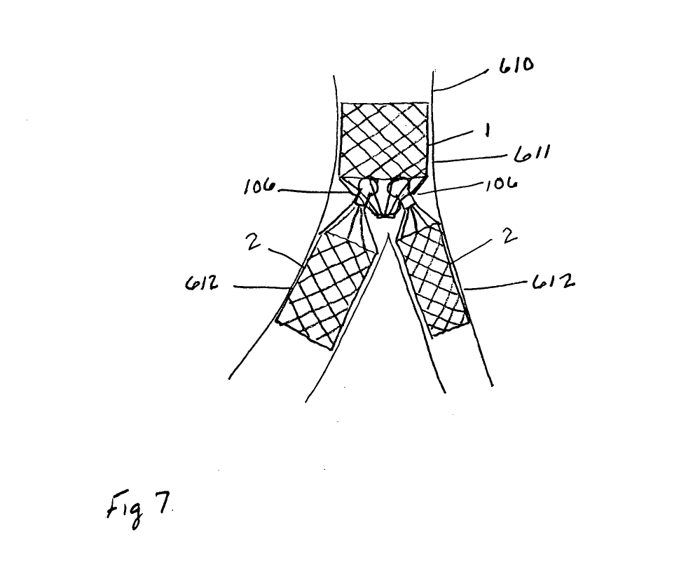

The application to bifurcated vessels (610) may require two or more main bodies (2) for treatment of two or more damaged vessels is shown in FIG. 7. Anchor body (1) is deployed above the bifurcation in a region of relatively healthy tissue (611) that can support the necessary anchor load. Anchor body in this case has two or more straight tube sections (106) in order to accommodate two or more main bodies (2). The connection between each main body (2) and anchor body (1) forms a connection similar to a ball joint as has been described. Each main body (2) is deployed to cover the region of damaged tissue (612).

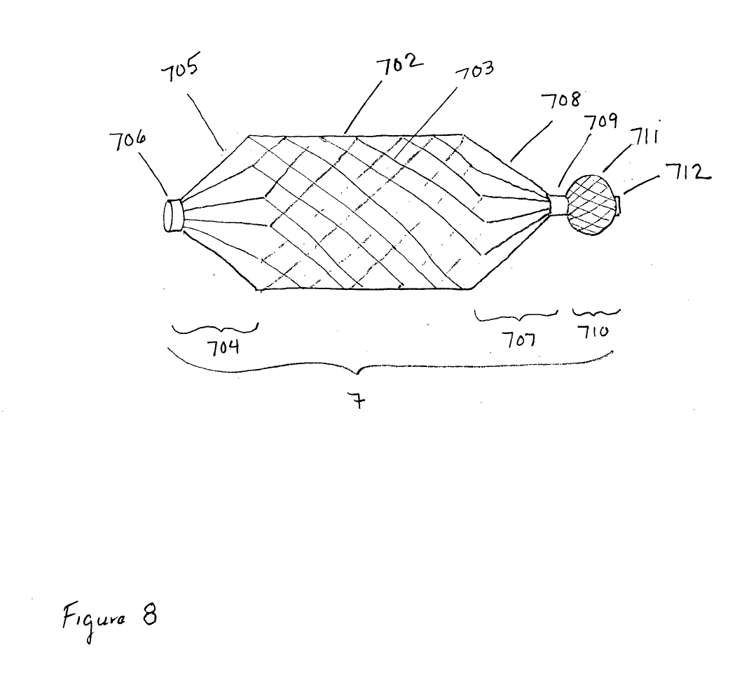

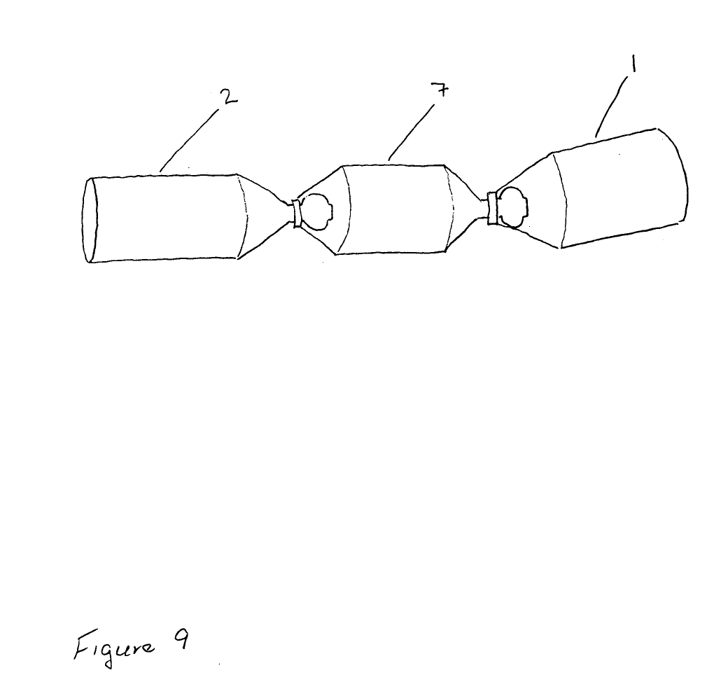

In an alternate embodiment, an intermediate anchor body (7) is shown in FIG. 8, It consists of a long cylindrical section (702) formed of a lattice of thin diagonal members (703) that can expand in the radial direction and transitions at its first end to a short tapered section (704). The tapered section (704) is made of thin longitudinal members (705) and terminates in a straight tube section (706) of reduced diameter. At the second end of said intermediate anchor body, cylindrical section (702) transitions to a short tapered section (707). Tapered section (707) is made of thin longitudinal members (708) and terminates in an intermediate tube section (709) of reduced diameter. The intermediate tube section (709) transitions to a partially expanded spherical section (710) made of a lattice of slender diagonal members (711) that can be expanded in the radial direction. Spherical section (710) transitions into an end straight tube section (712). This intermediate anchor body allows chaining multiple anchor bodies together where the connection between each anchor body substantially acts as a ball joint allowing relative rotation between bodies and finally attaches to a main body as previously described as shown in FIG. 9. It is also possible to attach multiple main bodies in this manner.

The foregoing description of the present invention describes an apparatus and method for excluding aneurysms occurring in the thoracic or abdominal aorta or for treating occlusive disease in the peripheral vasculature. It should be understood however, that the methods and apparatus of the present invention are equally applicable elsewhere in the human body where it is desired to repair a bifurcated vessel or organ, or to treat highly tortuous or angulated lumens. While preferred illustrative embodiments of the present invention are described above, it will be obvious to one skilled in the art that various changes and modifications may be made therein without departing from the invention and it is intended in the appended claims to cover all such changes and modifications which fall within the true spirit and scope of the invention.

What is claimed is:

1. An endoluminal prosthesis device comprising:
an anchor body with a first end and a second end and formed from a radially expandable lattice of thin diagonal members to engage the lumen,
a region of reduced radial expansion at the first end of the anchor body,
a main body formed from a radially expandable lattice of thin diagonal members to treat the lumen,
a means for engaging the main body to the anchor body at an area of reduced radial expansion on said anchor body.

2. An endoluminal prosthesis device comprising:
an anchor body formed from a long cylindrical section having a first end and a second end,
a tapered section at the first end of said anchor body,
said tapered section terminating in a straight tube section of reduced diameter compared to the cylindrical section,
a long cylindrical section on said main body having a first end and a second end,
a tapered section at the second end of said main body,
a partially expanded bulge section attached to said tapered section,
said expandable region being trapped within the necked region of the anchor body when deployed within vasculature such that the connection between the main body and anchor body substantially allows rotation of the main body relative to the anchor body but does not allow substantial relative translation.

3. The device of claim 2 wherein the anchor body and the main body are made from a self expanding shape memory material such as nitinol.

4. The device of claim 2 wherein the anchor body and the main body are made of a material that expands through elastic recovery such as stainless steel or nitinol.

5. The device of claim 2 wherein the anchor body and the main body expand to different diameters.

6. The device of claim 2 wherein said anchor body and said main body expand to substantially similar diameters.

7. The device of claim 2 wherein said anchor body has a long axis, and said main body has a long axis, said axes not necessarily being collinear after deployment.

8. The device of claim 7 wherein the long axis of said anchor body and the long axis of said main body have relative angular orientations that vary with time.

9. The device of claim 2 wherein the expandable bulge section is substantially spherical in shape, thus accommodating any rotational orientation about the long axis of the device.

10. The device of claim 2 wherein the bulge section in the main body and the tapered section in the anchor body are oval in shape thus accommodating a set angular orientation between the anchor body and the main body.

11. The device of claim 8 where radiopaque markers are located on the anchor body and the main body to establish the angular orientation between the two bodies.

12. The device of claim 2 wherein the Anchor body has two or more regions of reduced diameter in order to capture multiple main bodies.

13. The device of claim 2 wherein the main body is a stent.

14. The device of claim 2 wherein the main body is a stent-graft.

15. The device of claim 2 wherein the main body is a filter.

16. The device of claim 2 wherein the contact region between the two bodies is coated with a low friction material such as PTFE, UHMWPE, or Polyethelene.

17. A method of treating damaged vasculature using an endoluminal prosthesis comprising the steps of:
Placing an anchor body portion of the prosthetic device in a region of relatively healthy tissue within the vasculature proximal to the tissue region to be treated,
placing a main body so that it engages with said anchor body at a region of reduced diameter on said anchor body and said main body so that said main body is located at the region of vasculature to be treated.

18. The method of claim 17 further comprising:
Placing the anchor body within a catheter system and then deploying to the specified region of vasculature,
Placing the main body within the catheter and deploying to engage the anchor body at the region of vasculature to be treated.

19. The method of claim 17 further comprising:
Placing the anchor body and the main body within the catheter together in an orientation compatible with simultaneous placement and deployment within the vasculature.

20. A method of treating damaged vasculature using an endoluminal prosthesis comprising the steps of:
Placing a first anchor body portion of the prosthetic device in a region of relatively health tissue within the vasculature proximal to the tissue region to be treated,
placing a second anchor body portion of the prosthetic device so that it engages with said first anchor body at a region of reduced diameter on said anchor body,
placing a main body portion so that it engages with said second anchor body at a region of reduced diameter on said anchor body, so that said main body is located at the region of vasculature to be treated.
